(19) United States
(12) Patent Application Publication
(10) Pub. No.: US 2019/0017987 A1
(43) Pub. Date: Jan. 17, 2019

(54) PERFORMING ONE OR MORE ANALYSES ON A THIN LAYER OF BIOLOGIC FLUID USING OPTICALLY RESPONSIVE CHEMICAL SENSORS

(71) Applicants: Robert A. LEVINE, Guilford, CT (US); Stephen C. WARDLAW, Lyme, CT (US)

(72) Inventors: Robert A. LEVINE, Guilford, CT (US); Stephen C. WARDLAW, Lyme, CT (US)

(21) Appl. No.: 16/070,166

(22) PCT Filed: Oct. 14, 2016
(86) PCT No.: PCT/US16/56965

§ 371 Date: Jul. 13, 2018

Related U.S. Application Data
(60) Provisional application No. 62/279,451, filed on Jan. 15, 2016.

(51) Int. Cl. G01N 33/487; G01N 21/64; G01N 21/78

(52) U.S. Cl. CPC G01N 33/487; G01N 21/6428; G01N 2021/7786; G01N 21/78; G01N 2021/6434; G01N 21/6489

(57) ABSTRACT

A method and apparatus for analyzing a biologic fluid sample for at least one target analyte is provided. The method includes providing an analysis chamber having at least one optically responsive chemical sensor (ORCS), sample and calibration fluid regions, and at least one fluid separator that separates the sample and calibration fluid regions. A first portion of each sensor is disposed in the sample fluid region and a second portion of each sensor is disposed in the calibration fluid region. The ORCS is configured to optically respond in the presence of the target analyte and when interrogated with one or more predetermined wavelengths of light. The method further includes disposing at least one calibration fluid having target analyte in a known or ascertainable concentration in the calibration fluid region, and disposing the biologic fluid sample in the sample fluid region. The sample is analyzed using a first optical response from the first portion of the ORCS and a second optical response from the second portion of the ORCS.

This application claims priority to U.S. Patent Appln. Ser. No. 62/279,451 filed Jan. 15, 2016, which is herein incorporated by reference in its entirety.

### BACKGROUND OF THE INVENTION

### 1. Technical Field

The present disclosure relates to chemical analyses of a biologic fluid sample in general, and to chemical analyses of a biologic fluid sample using an optically-responsive chemical sensor (“ORCS”), in particular.

### 2. Background Information

In vitro point of care diagnostics can permit rapid evaluation of biologic samples at the office of a healthcare provider or at a remote location. Point of care (“POC”) diagnostics have the potential of increasing access to healthcare and the speed and efficiency at which healthcare can be administered. Some currently available POC type systems for chemical analysis utilize an electronic chip-based cartridge, which cartridge may perform anywhere from one to eight tests. Such a cartridge may include one or more ion specific electrodes (ISE). An ISE is a transducer (or sensor) that converts the activity of a specific ion dissolved in a solution into an electrical potential, which can be measured using various electrical sensing means. Typically, the cost of constructing and disposing a dedicated electronic chip within an analysis cartridge is relatively high, and the high cost of such a device can inhibit its use and thereby limit the POC benefits associated therewith. Additionally, the manufacture of the sensor chips requires a semiconductor fabrication facility (FAB), which is extremely costly and therefore limits the number of potential users of the technology, and thus limiting competition among manufacturers.

In some instances specific ions can be detected within a fluid sample using an ORCS that produces an output signal in the form of photons fluorescently emitted as a result of photometric excitation. In another instance, ORCS can be configured to change their color and/or color intensity, either transmitted or reflected. The accuracy of existing ORCSs can depend, however, on factors such as time, temperature and most importantly, variances in the manufacturing process. These factors inhibit the use of ORCS in a single-use point-of-care system.

Flow cytometry is another technology that can be used to perform chemical analyses on a biologic fluid sample. Flow cytometers are capable of multiplexing but are not capable of using whole undiluted blood. Another disadvantage of flow cytometers is that the sample flows past one or more sensors during analysis. Hence, the amount of time available for sensing is limited by the sample flow rate.

Embodiments of the present invention, illustrated in the attached figures, overcome these aforementioned problems, allowing the construction of a low-cost, practical photometric analysis system that can be implemented in a point-of-care configuration or in any location requiring low-cost analyses.

### SUMMARY OF THE INVENTION

According to an aspect of the present disclosure, a method of analyzing a biologic fluid sample for at least one target analyte is provided. The method includes: providing an analysis chamber having at least one optically responsive chemical sensor (ORCS) disposed on a substrate surface, a sample fluid region, a calibration fluid region, and at least one fluid separator fluidically separating the sample fluid region and the calibration fluid region. A first portion of the at least one ORCS is disposed in the sample fluid region and a second portion of the at least one ORCS is disposed in the calibration fluid region. The method further includes: disposing at least one calibration fluid that includes the target analyte in a known or ascertainable concentration in the calibration fluid region; disposing the biologic fluid sample in the sample fluid region; and using at least one light source to interrogate the first portion of the ORCS and the second portion of the ORCS with one or more predetermined wavelengths of light, using the at least one light detector to detect a first optical response from the first portion of the ORCS when interrogated and a second optical response from the second portion of the ORCS when interrogated; and using a processing unit having at least one processor to analyze the biologic fluid sample relative to the at least one target analyte using the detected first and second optical responses.

According to another aspect of the present disclosure, an apparatus for analyzing a biologic fluid sample for at least one target analyte is provided. The apparatus includes an analysis chamber, at least one light source and at least one light detector, and a processing unit. The analysis chamber has at least one optically responsive chemical sensor (ORCS) disposed on a substrate surface, a sample fluid region, a calibration fluid region, and at least one fluid separator fluidically separating the sample fluid region and the calibration fluid region. A first portion of the at least one ORCS is disposed in the sample fluid region and a second portion of the ORCS is disposed in the calibration fluid region. The processing unit has at least one processor. The processing unit is in communication with the at least one light source and the at least one light detector, and in communication with a memory device storing instructions. The instructions when executed cause the processing unit to: control the at least one light source to interrogate the first portion of the ORCS and the second portion of the ORCS with one or more predetermined wavelengths of light, and control the at least one light detector to detect a first optical response from the first portion of the ORCS when interrogated and a second optical response from the second portion of the ORCS when interrogated, and analyze the biologic fluid sample using the detected first and second optical responses.

According to another aspect of the present disclosure, an analysis chamber is provided. The analysis chamber includes at least one optically responsive chemical sensor (ORCS), a sample fluid region, a calibration fluid region, and at least one fluid separator. The ORCS is disposed on a substrate surface. The ORCS is configured to optically respond in the presence of a target analyte and when interrogated with one or more predetermined wavelengths of light. The at least one fluid separator fluidically separates the sample fluid region and the calibration fluid region. A first portion of the at least one ORCS is disposed in the sample fluid region and a second portion of the ORCS is disposed in the calibration fluid region.

In a further embodiment of any of the foregoing aspects, the at least one ORCS includes a plurality of optodes configured to optically respond in the presence of the target analyte while being interrogated with the one or more predetermined wavelengths of light. The plurality of optodes are substantially uniformly distributed in both the first and second portions of the ORCS.

In a further embodiment of any of the foregoing aspects and embodiments, each of the optodes includes ionophores that selectively interact with the target analyte, chomoionophores that optically respond as a function of protonation, and a matrix.

In a further embodiment of any of the foregoing aspects and embodiments, the first and second optical responses are a change in at least one of fluorescent emission, absorbance or reflectance.

In a further embodiment of any of the foregoing aspects and embodiments, the first and second optical responses include the optodes emitting fluorescent emissions at a first wavelength in the absence of the target analyte when interrogated by the one or more predetermined wavelengths of light, and the optodes emitting fluorescent emissions at a second wavelength in the presence of the target analyte when interrogated by the one or more predetermined wavelengths of light, which second wavelength is different from the first wavelength. In some embodiments, the optical response may include a change in fluorescent intensity rather than a change in wavelength.

In a further embodiment of any of the foregoing aspects and embodiments, the at least one light detector produces first signals indicative of the first optical response and second signals indicative of the second optical response, and the processing unit analyzes the biologic fluid sample relative to the at least one target analyte using the first signals and the second signals.

In a further embodiment of any of the foregoing aspects and embodiments, the analysis chamber includes a first ORCS that includes a plurality of first optodes selectively sensitive to a first target analyte, which plurality of first optodes are substantially uniformly distributed in both the first and second portions of the first ORCS, and includes a second ORCS that includes a plurality of second optodes selectively sensitive to a second target analyte, which plurality of second optodes are substantially uniformly distributed in both the first and second portions of the second ORCS. The first target analyte is different in type from the second target analyte.

In a further embodiment of any of the foregoing aspects and embodiments, the interrogating and detecting are performed a plurality of times prior to a reaction between the target analyte and the ORCS reaching equilibrium, and the analyzing the data provides a kinetic analysis or a predictive end-point analysis.

The present invention, which includes using one or more ORCS for chemical analyses, provides several advantages over the presently available chemical, photometric, electrochemical, potentiometric, and automated immunoassay methodologies, including reduced cost and increased simplicity. For example, in regards to those methodologies that utilize an electronic chip, the cost of constructing and disposing a dedicated electronic chip for analysis is very high, especially considering that millions of such analyses may be performed every week. The present invention does not require such a chip. Relatively speaking, the cost to produce an ORCS (e.g., optodes in particulate or bulk form) is comparatively very low. The production of large numbers of electronic chip-based cartridges, each of which may perform one to eight tests is high. The cost of a cartridge as is contemplated under the present invention, which cartridge can hold multiple families of ORCSs, is comparatively low since the “brains” are in the optical reader and in the mass producible ORCSs.

The foregoing features and elements may be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIG. 1 is a diagrammatic partial cross-sectional view of an analysis chamber embodiment.

FIG. 2 is a diagrammatic partial perspective view of an analysis chamber embodiment.

FIG. 3 is a diagrammatic view of analysis chamber embodiments including a tape substrate.

FIG. 4 is a diagrammatic view of an analysis cartridge embodiment.

FIG. 5 is a diagrammatic view of the analysis cartridge embodiment shown in FIG. 4, illustrating and analysis device reader.

FIG. 6 is a diagrammatic view of an analysis chamber embodiment.

FIG. 7 is a diagrammatic view of an analysis device embodiment.

FIG. 8 is diagrammatic partial view of an analysis chamber embodiment.

FIG. 9 is a flow chart illustrating method embodiments of the present disclosure.

### DETAILED DESCRIPTION OF PREFERRED EMBODIMENTS

Aspects of the present disclosure include a system, apparatus, and method for performing one or more qualitative and/or quantitative analyte analyses on a single biologic fluid sample in an analysis chamber. In some embodiments, multiple analyses may be performed on a single sample simultaneously. The multiple analyses may include analyses to determine the presence or absence of different target analytes (or chemical environments), and or may include multiple analyses to determine the presence of absence of the same target analyte; e.g., in different concentrations. In some embodiments, an analysis may be performed as an equilibrium type analysis. In some embodiments, the analysis may be performed as a kinetic type analysis. Aspects of the present disclosure utilize one or more ORCSs disposed within an analysis chamber configured to quiescently hold a thin layer of biologic fluid sample. The ORCSs may function as fluoresence emitting and/or optical density or reflectance modulating, ion or chemical-specific sensors. Non-limiting examples of biologic fluid samples that may be analyzed using aspects of the present disclosure include blood (e.g., substantially undiluted whole blood), urine, cerebrospinal fluid, joint fluid, and other body fluids. Specific analytes that may be quantified with the present disclosure include, but are not limited to: sodium, potassium, chloride, calcium, bicarbonate, glucose, urea creatinine and ligand-based analyses. The analysis chamber includes a calibration region for receiving one or more calibration fluids and a sample region for receiving the biologic fluid sample to be analyzed. In some embodiments, as will be described below, the present disclosure includes an instrument that is configured to accomplish one or more of: a) selectively interrogate the analysis chamber containing the ORCS, calibration fluid(s), and biologic fluid sample with light (e.g., at predetermined wavelengths)and sense light emanating from the analysis chamber (e.g., fluorescently emitted light) and/or light absorbed within or reflected from the analysis chamber; and b) determine the presence or absence of an analyte (and/or) provide a quantitative measurement of an analyte within the biologic fluid sample based upon said the relative responses of the sample and calibration regions.

For the purpose of clarity, the term “ORCS” as used herein refers to a sensor composition (which may include one or more optodes) that produces an optical response in response to a change in the chemical environment to which the ORCS is exposed; e.g., the ORCS is selectively sensitive to the change in chemical environment. The “change” in the chemical environment may be the introduction of a target analyte into the aforesaid environment as will be described below; e.g., the ORCS may be selectively sensitive to the presence of the target analyte. The term “optical response” as used herein refers to a change in a sensible property of light by the ORCS that occurs when the ORCS is subjected to the change in chemical environment and illuminated by one or more predetermined wavelengths of light. Non-limiting examples of optical responses include a change in wavelength of fluorescently emitted light, a change in the intensity of a given wavelength of light, a change in absorbance of light, etc.

The ORCS may itself be configured as an optode, or may be configured to include a plurality of particulate optodes, or may contain a reagent(s) that reacts with a target analyte (or reacts in response to a chemical environment change) to alter the color or color intensity or absorbance of the aforesaid reagent(s). The present disclosure is not limited to any particular optode configuration or composition. In some embodiments, for example, an optode may include an ionophore and a chromoionophore disposed within a matrix. The optode is not limited to these particular elements and may optionally include additional elements such as a fluorescent semiconductor nanocrystal (sometimes referred to as a “quantum dot”) and additives that enhance the functionality, manufacturability, durability, etc. of the ORCS.

An ionophore included within an optode may be a compound, typically an electrically neutral compound, that associates (e.g., forms a complex, chelate, or other non-covalent association) with a target analyte (e.g., a target ion), and is selective for the target analyte relative to other analytes.

A chromoionophore included within an optode may be an ionophore that changes its optical properties (e.g., fluorescence or absorbance) in the visible spectrum depending on the state of complexation. A chromoionophore may, for example, be a proton-sensitive dye that changes absorbance (and fluorescence in many cases) depending on the degree of protonation, although chromoionophores that change absorbance in response to other ions can also be used. The chromoionophore may be highly lipohilic to inhibit it from leaching out of the matrix.

The matrix may be a material (e.g., a polymeric material) used to combine the ionophores and chromoionophores or other reactive agents into a collective form, one that does not adversely affect the analysis at hand (e.g., chemically adversely affect), and one that does not adversely impede detection of any optical response associated with an optode or reactive agent (and therefore an optical response collectively associated with the ORCS). In some embodiments, the matrix is configured to allow target analyte within the fluid sample or calibration fluid to diffuse through the matrix to reach the ionophore (and in some instances chromoionophore) elements disposed within the matrix. In some embodiments, the matrix may also be operable to block elements or materials present within the sample (e.g., blood cells, platelets, proteins or the like) from entering the optode and thereby potentially negatively inhibiting the passage of analyte within the optode. The relative percentages of the ionophore, chromoionophore, and matrix within the optode are chosen to suit the requirements of the application at hand, and may, for example, be determined by experimentation for each analyte to optimize optode performance. Non-limiting examples of acceptable polymeric matrix materials consist essentially of polyvinyl chloride (PVC), polymethyl methacrylate (PMMA) and decyl methacrylate or copolymers or any combination thereof, or may include gels, dried or not, such as Phytogel or agarose.

In those embodiments of the present disclosure wherein the target analyte is an ionic analyte, the chromoionophore changes state in response to proton concentration (i.e., the protonated chromoionophore is one state while the deprotonated chromoionophore is a second state), and the ionophore selectively associates with the target ionic analyte. Once the ionophore associates with a cationic analyte (e.g., Na⁺ associates with a Na⁺-selective ionophore), for example, protons are displaced from the optode to equilibrate charge, altering the state of the chromoionophore. The altered state of the optode indicates the state of the chromoionophore, which in turn correlates to the presence and/or concentration of the ionic analyte.

Examples of optode compositions that may be used with the present disclosure are disclosed in Fluorescent Sensors for the Basic Metabolic Panel Enable Measurement with a Smart Phone Device Over the Physiological Range, Awquatty et al., Analyst, 2014, 139, 5230, and provided below. The present disclosure is not, however, limited to these particular examples.

### Sodium Optode Composition:

An optode configured to sense sodium within a sample may be made in acetone, and prepared to include the following components: 25 mg ml⁻¹ poly(caprolactone) ˜14 000 Mₙ, 8.3 mg ml⁻¹ Acetyl-tri-n-hexyl citrate (Citroflex A6) (Vertellus, Indianapolis, Ind.), 0.67 mg ml⁻¹ Sodium Ionophore X (NaIX), 0.33 mg Sodium Tetrakis-[3,5-bis(trifluoromethyl) phenyl] Borate (NaTFPB), 0.167 mg ml⁻¹ Chromoionophore III (CHIII), and 0.067 mg ml⁻¹ Octadecyl Rhodamine b Chloride (rhodC18) (Invitrogen).

### Potassium Optode Composition:

An optode configured to sense potassium within a sample may be made in acetone, and prepared to include the following components: 25 mg ml⁻¹ Poly(caprolactone) ˜14 000 Mₙ, 8.3 mg ml⁻¹ Citroflex A6, 1 mg ml⁻¹ Potassium Ionophore III, 0.33 mg ml⁻¹ Potassium Tetrakis-[3,5-bis(trifluoromethyl) phenyl] Borate (KTFPB), 0.167 mg ml⁻¹ CHIII, and 0.067 mg ml⁻¹ rhodC18.

### Calcium Optode Composition:

An optode configured to sense calcium within a sample may be made in acetone, and prepared to include the following components: 25 mg ml⁻¹ poly(caprolactone) ˜14 000 Mₙ, 8.3 mg ml⁻¹ Citroflex A6, 0.33 mg ml⁻¹ Calcium Ionophore II, 0.33 mg ml⁻¹ KTFPB, 0.167 mg ml⁻¹ CHIII, and 0.067 mg ml⁻¹ rhodC18.

### Chloride Optode Composition:

An optode configured to sense chloride within a sample may be made in tetrahydrofuran, and prepared to include the following components: 60 mg ml⁻¹ poly(vinyl chloride), 120 mg ml⁻¹ 2-nitrophenyl octyl ether, 4 mg ml⁻¹ Chloride Ionophore IV, 2 mg ml⁻¹ KTFPB, 1 mg ml⁻¹ CHIII, and 0.4 mg ml⁻¹ rhodC18.

### pH Optode Composition:

In those applications wherein the pH of a fluid sample may affect analytical results a pH optode may be included. An pH optode configured to sense the pH of a fluid sample may be made in acetone and prepared to include the following components: 25 mg ml⁻¹ poly(caprolactone) ˜14 000 Mₙ, 8.3 mg ml⁻¹ Citroflex A6, 0.083 mg ml⁻¹ KTFPB, 0.167 mg ml⁻¹ CHIII, and 0.067 mg ml⁻¹ rhodC18.

In some embodiments, non-specific optodes may be used as detectors, with the specific chemistry taking place within the matrix. For example, for a glucose test, a glucose oxidase reaction in the matrix could be “read” by a generic redox-sensitive optode. In another examples, reagents within the matrix can react with the analyte to form a detectable colored (or fluorescent) product, a common example of such being a test strip for glucose.

As stated above, the above described optode compositions are provided for illustrative purposes, and the present disclosure should not therefore be interpreted as being limited to these particular compositions. U.S. Pat. Nos. 8,114,662; 8,263,358; 8,268,567; 8,470,300; and 8,765,458, each of which is hereby incorporated by reference in its entirety, also disclose materials that may be utilized within an ORCS.

As indicated above, optodes included within a present disclosure ORCS may assume a variety of different forms. For example, an ORCS may include a number of optodes configured in particulate form. The term “particulate”, as used herein refers to an optode that is very small in size relative to the ORCS, but is itself configured to produce an optical response in response to a change in its chemical environment. Each particulate optode may include ionophores, chromoionophores, and a matrix as described above. ORCSs that include particulate optodes typically include a relatively large number of particulate optodes, and the particulate optodes collectively produce a sensible optical response when exposed to the target analyte. ORCSs that include particulate optodes may additionally include a carrier medium that holds the particulate optodes in a substantially uniform distribution. The carrier medium does not adversely affect the analysis at hand (e.g., chemically adversely affect) or adversely impede detection of any optical response associated with the particulate optodes (and therefore an optical response collectively associated with the sensor), and allows the target analyte within the fluid sample or calibration fluid to reach the particulate optodes. The carrier medium may also be configured to facilitate the process used to deposit the ORCS on to the surface of a substrate; e.g., a printing process, an extrusion process, etc.

The present disclosure is not limited to any particular type of particulate optode configuration. An example of an acceptable particulate optode configuration is one in which ionophores and chromoionophores are disposed within a polymer matrix collectively formed as a particulate. As indicated above when the particulate optode is exposed to an environment containing target analytes, the target analytes are drawn into the particulate optode where they bind with the target selective ionophores. To maintain charge neutrality within the optode, protons disassociate with the chromoionophores and diffuse out of the optode, thereby altering the photometric state of the optode. Another example of an acceptable particulate optode configuration is one in which the optode is formed similar to a micelle type particle wherein chromoionophores are disposed on an exterior surface of the particle surrounding a core formed of the matrix material and ionophores. The mechanism of altering the photometric state of the optode is the same as that disclosed above. ORCS containing a plurality of particulate sensors may sometimes be referred to as a “bulk optode”

The characteristics (e.g., individual size, concentration, etc.) of particulate optodes within an ORCS may vary to satisfy the requirements of the specific assay at hand. The particulate optode characteristics may be determined by experimentation for each target analyte to optimize the ORCS performance (e.g., signal to noise ratio). For those target analytes that may have a wide biologic range, different ORCS configurations may be used for the same target analyte; e.g., the ORCSs within a chamber may include more than one ORCS directed to a particular target analyte (e.g., a first ORCS with a first concentration of particulate optodes selective to the target analyte, and a second ORCS with a second concentration of particulate optodes also selective to the target analyte, which second concentration is greater than the first concentration). The two ORCSs provide the analysis chamber with a wider dynamic range for the assay at hand. In this regard, aspects of the present disclosure may be described as providing a multi-point (e.g., a two-point) analysis device that can accommodate a larger range of analyte concentrations and still produce useful data.

In alternative embodiments, an ORCS itself may be foil led as a single optode comprising the above-described ionophores and chromoionophores disposed within a polymer matrix.

The term “analysis area” as used herein refers to an area of the chamber containing one or more ORCSs wetted by the biologic fluid sample and/or an area of the chamber containing the aforesaid one or more ORCSs wetted by one or more calibration fluids.

The term “analysis period” as used herein refers to a time interval between a first point in time when the biologic fluid sample is dispensed into the chamber and a second point in time when sample data acquisition is acquired.

The term “calibration fluid” as used herein refers to a fluid containing at least one analyte that is the same as or similar to the analyte targeted within the analysis. As will be explained below, in some embodiments a single calibration fluid can be used that includes a number of different types of analytes, which number is equal to or greater than the number of different target analyte types to be investigated within the sample. The analytes within the calibration fluid(s) are at a known or ascertainable concentration and preferably are a concentration that is substantially equal to an average concentration value of the target analytes thought to be present within the sample to be analyzed and additional higher or lower than average concentration of target analytes preferably at clinical decision levels, may be added in additional calibration regions.

The term “biologic fluid” as used herein means any biologic fluid available or obtained from a biologic organism including all animals and plants, including biologic fluids that may or may not contain particulate matter, such as whole blood. As stated above, non-limiting examples of biologic fluid samples that may be analyzed using aspects of the present disclosure include blood (e.g., substantially undiluted whole blood), urine, cerebrospinal fluid, joint fluid, and other body fluids. Specific analytes that may be quantified with the present disclosure include, but are not limited to: sodium, potassium, chloride, calcium, bicarbonate, glucose, urea creatinine and ligand-based analyses.

The term “chemical analysis” as used herein means the qualitative and/or quantitative analysis of chemical analytes such as ions, molecules such as glucose, urea, creatinine, hormones, enzymes, tumor markers, antibodies and nucleotides, as well as prions and viral particles or bacteria or protozoa that can be detected by selective detection of their chemical nature, either intact or disrupted.

The term “determination of the concentration of a chemical analyte” as used herein refers to a concentration determination of a chemical analyte by measurement of the concentration without measurement or knowledge of the volume of the sample other than the range of possible contents of the chamber.

The term “equilibrium assay” as used herein means an assay that is completed at the end point of the assay when the signal is stable.

The term “predictive end-point calculation” as used herein shall mean the repetitive reading of a signal(s) from the ORCS during the assay time and using computational means to fit the response over time to calculate what the result would be if the reaction proceeded to final equilibrium.

The term “kinetic assay” as used herein means an assay that is performed repetitively during the assay time and the slope or mathematically modeled time course of the signal as well as its intensity is used to calculate the concentration of the target analyte(s).

The present disclosure may be implemented using a variety of different analysis chamber configurations, and is not therefore limited to any particular configuration. The analysis chamber includes at least one planar substrate that is sufficiently optically transparent to permit light to pass through to interrogate the biologic fluid sample, calibration fluid(s), and ORCS residing within the chamber. In some embodiments, the chamber may be configured to allow light to be transmitted through the entire chamber. The planar substrate may be configured to be part of a cartridge, or may be a portion of a tape (e.g., that can be wound and unwound from reels), or manufactured from a tape (described below). The analysis chamber includes at least one calibration region configured to receive one or more calibration fluids, and at least one sample region configured to receive the biologic sample fluid to be analyzed.

FIG. 1 illustrates an analysis chamber 10 embodiment that includes a planar substrate 12 and a cover sheet 14, at least one of which is sufficiently optically transparent as described above. The planar substrate may comprise a variety of materials (e.g., a polymeric material, glass, etc.) and is typically sufficiently rigid to support a fluid sample residing on a surface of the substrate without appreciably bending due to the weight of the fluid sample. The cover sheet 14 may be the same material and configuration as the planar substrate, but is not so required. The cover sheet 14 may be any material and/or configuration that can be disposed on top of the sample to substantially enclose the sample relative to the planar substrate 12, and one that will preferably not inhibit capillary fluid flow between the substrate 12 and the cover sheet 14. In some embodiments, the cover sheet 14 may be configured to prevent or substantially impede evaporation of one or more constituents from the biologic fluid sample and/or the calibration fluid for a period of time useful for analysis. The planar substrate 12 has an inner surface 12A and an outer surface 12B. The cover sheet 14 has an inner surface 14A and an outer surface 14B. The inner surface 12A of the planar substrate 12 faces the inner surface 14A of the cover sheet 14. The chamber 10 is preferably configured such that the substrate 12 and the cover sheet 14 are separated from one another by a distance 16 (hereinafter referred to as the “chamber height” or chamber “through-plane thickness”, which may be defined by a bisecting line representing the shortest distance between the respective inner surfaces 12A, 14A) to permit the biologic fluid sample to be introduced and quiescently held between the inner surfaces of the substrate 12 and cover sheet 14. The chamber height is preferably such that a biologic fluid sample and a calibration fluid can be drawn into the void between the substrate and the cover sheet by capillary action. The chamber height is not, however, limited to any particular dimension and need not be precise since, as will be discussed below, signals associated with an ORCS (and in some embodiments the optode(s) contained therein) are related to the chemical target analyte concentrations local to the respective ORCS, and the composition and geometry of the respective ORCS; i.e., the signals are independent of the volume of the sample. For most analyses, a chamber height in the range of about 5 to 500 microns is useful, and a chamber height of about 50 microns is believed to be particularly useful. The planar substrate 12 and the cover sheet 14 may be parallel to one another.

The separation distance 16 (i.e., the “through-plane thickness”) between the planar substrate inner surface 12A and the cover sheet inner surface 14A may be established by various different means. For example, in some embodiments if the planar substrate 12 and the cover sheet 14 are sufficiently rigid, it may be adequate to position the substrate 12 and the cover sheet 14 a distance away from one another around their perimeters. In other embodiments, it may be desirable to include one or more physical elements 18 (e.g., “spacers”) disposed between and in contact with the respective inner surfaces 12A, 14A. A spacer 18 may be integral with the substrate 12 or the cover sheet 14, or may be independent of both. As indicated above, the chamber height 16 (and therefore the related spacer dimension) need not be precise. A spacer 18 may be any element that extends between the inner surfaces 12A, 14A and is operable to space the substrate 12 and cover sheet 14 apart from one another.

In some embodiments, the analysis chamber 10 may include a single planar substrate 12 in which case the biologic fluid sample, the calibration fluid, and ORCS (including optode(s)) may be deposited on a surface of the substrate 12 for analysis.

Referring to FIG. 3, in some embodiments the analysis chamber 10 may be manufactured using a single polymer tape, or using a first polymer tape and a second polymer tape. A plurality of chambers 10 may be manufactured by depositing ORCSs 20 on a surface of the tape and creating a fluid separator 22 that separates each ORCS 20 into two portions; e.g., a lengthwise extending ORCS 20 sectioned by a widthwise extending fluid separator 22 as will be described below. In those chamber 10 embodiments that include both a first and second tape, the one or more ORCS 20 and fluid separator 22 may be formed on a surface of the first tape, and then the second tape lacking the ORCS may be laid over the first tape: i.e., the ORCS and fluid separator are disposed between the tapes. During manufacturing, the deposition process is repeated along the length of the tape, and the tape may be later cut in between the sensor/fluid separator configurations to form individual analysis chambers. FIG. 3 diagrammatically illustrates a tape 24 with an initial chamber 10A bearing a plurality of ORCS 20 deposited in a linear fashion. The next three chambers 10B, 10C, 10D each including ORCSs 20 and a fluid separator 22 extending across the ORCSs 20; e.g., the fluid separator 22 for each chamber is disposed to separate each ORCS into two portions. The last chamber 10E is separated from the tape. An advantage of creating analysis chambers 10 in this manner is that each ORCS 20 is deposited in a single act (i.e., at one point in time, from one source, etc.) and the fluid separator 22 splits the respective ORCS 20 into two sensor portions, each compositionally the same. This type of ORCS manufacturing process (i.e., wherein an ORCS 20 is deposited in a single act (i.e., at one point in time, from one source, etc.) and the fluid separator 22 splits the respective ORCS 20 into two sensor portions, each compositionally the same) is preferably used to create the ORCS regardless of the particular chamber 10 or cartridge 26 configuration. Producing an analysis chamber 10 in this manner decreases any variability that might otherwise exist if the two portions of a particular ORCS 20 were manufactured by separate processes (e.g., even ORCS material from a single source may vary to some degree as a function of the manufacturing run if, for example, the source material was not 100% uniformly mixed or deposited, etc.) By creating a single ORCS 20 and fluidically splitting it into two portions, it is believed that manufacturing variances for each ORCS 20 will be minimized, and as is described herein, it permits the analyses of the sample region and calibration region of a particular ORCS to be performed on the same ORCS, albeit different sections of the ORCS.

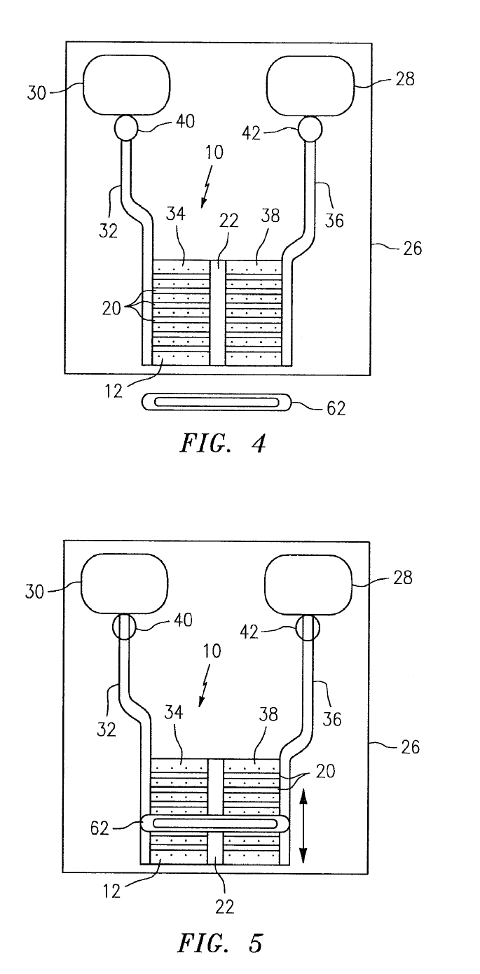

In some embodiments of the present disclosure, the analysis chamber 10 is an element of a disposable cartridge 26. In addition to the analysis chamber 10, the cartridge 26 may include other elements useful in performing an analysis such as a reservoir(s) 28 for holding one or more calibration fluids, at least one reservoir 30 for holding a biologic fluid sample, and optionally one or more elements configured to control fluid flow from the aforesaid reservoirs. For example, FIGS. 4 and 5 diagrammatically illustrate a cartridge 26 having a biologic fluid sample reservoir 30 and a calibration fluid reservoir 28. A first fluid passage 32 fluidically connects the biologic fluid sample reservoir 30 to a first region of the analysis chamber 10 (i.e., the sample region 34 located on a first side of a fluid separator 22), and a second fluid passage 36 fluidically connects the calibration fluid reservoir 28 to a second region of the analysis chamber 10 (i.e., the calibration region 38 on a second side of the fluid separator 22, opposite the first side). In the embodiment shown in FIGS. 4 and 5, the cartridge 26 further includes a first fluid control element 40 disposed to control fluid flow within the first fluid passage 32, and a second fluid control element 42 disposed to control fluid flow within the second fluid passage 36. The present disclosure is not limited to any particular type of fluid control element; e.g., acceptable fluid control elements include valves, capillary stops and rupturable membranes. Fluid flow from the respective reservoirs 28, 30 through the passages 32, 36 may be accomplished by a variety of different techniques; e.g., by capillary action, or by selectively applied motive force. As indicated above, fluid flow into the respective chamber regions 34, 38 may be accomplished by a capillary action, but other motive forces may be utilized alternately. The exemplary cartridge 26 shown and described represents a non-limiting example of a cartridge. The cartridge 26 may be configured to retain one or both of the calibration fluid and the sample fluid as a sealed container; e.g., once the analysis of the sample is performed, the sealed cartridge may safely contain the analysis materials and thereby allow the cartridge to be disposed of properly with little or no risk of biohazard material leakage.

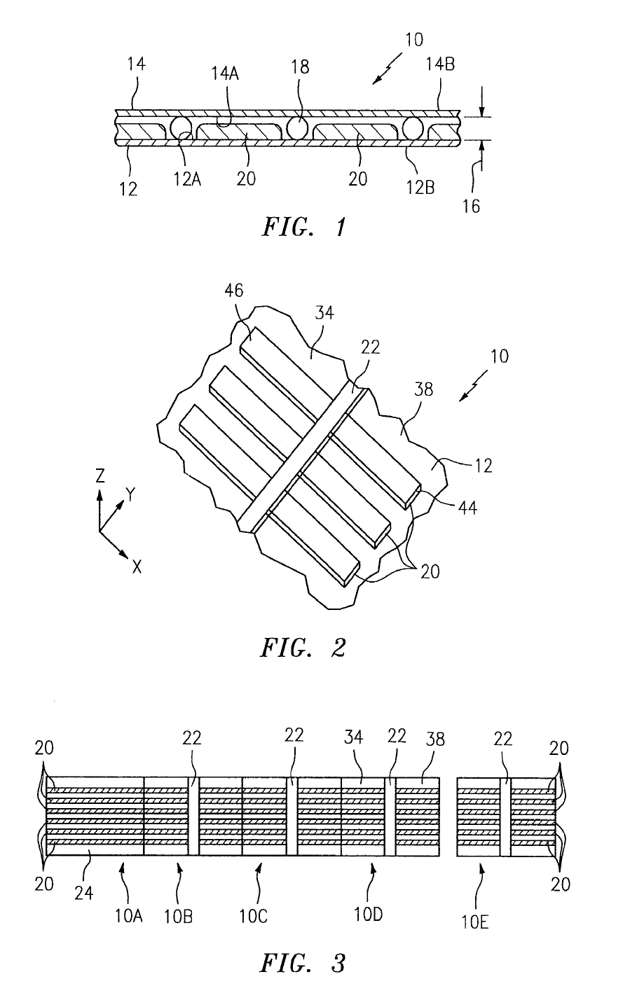

The ORCS(s) 20 utilized within the present disclosure are arranged on a substrate (i.e., a chamber substrate 12) in a manner that facilitates the performance of analyses. For example, in some embodiments one or more ORCS 20 may be formed as lengthwise extending strips deposited on a substrate surface. FIGS. 4 and 5 diagrammatically illustrate an analysis chamber 10 having a plurality of ORCSs 20 arranged as lengthwise extending strips disposed on a chamber substrate surface. Referring to FIG. 2, each of the ORCS strips 20 may be described as having a length, a width, and a height; e.g., in terms of orthogonal axes, the length of an ORCS strip may extend along an X-axis, the width of the strip may extend along the Y-axis, and the height (also referred to as “thickness”) of the strip may extend along the Z-axis. The ORCS strips 20 extend lengthwise from a first end 44 to a second end 46. Each ORCS strip 20 preferably has a substantially constant cross-sectional geometry (e.g., in the Y-Z plane) for substantially the entire length of the strip 20. Each ORCS strip 20 may be configured to sense a different chemical analyte (e.g., Potassium (K⁺), Sodium (Na⁺), Chloride (Cl⁻), Bicarbonate (HCO₃₋), Calcium (Ca²⁺), etc.).

Referring to FIGS. 4-6, a fluid separator 22 (e.g., a strip of hydrophobic material, a physical configuration, or any element operable to prevent fluid passage) is disposed between the first and second ends 44, 46 of the ORCS strips 20 and extends in a manner that separates each ORCS strip 20 into a first portion 20A and a second portion 20B; e.g., extends widthwise relative to lengthwise extending ORCS strips 20. Fluoropel™ (Cytonix LLC, MD, USA) is an example of a hydrophobic material that can be used. The hydrophobic material may be applied to the substrate inner surface 12A, and/or the cover sheet inner surface 14A to arrest capillary fluid flow. The fluid separator 22 separates each sensor strip 20 into two portions 20A, 20B and collectively forms a calibration region 38 on one side of the fluid separator 22 and a sample region 34 on the opposite side of the fluid separator 22 (i.e., a portion 20B of each ORCS strip 20 is disposed in the calibration region 38 and the other portion 20A of each ORCS strip 20 is disposed in the sample region 34). The fluid separator 22 allows one or more calibration fluids to reside within the calibration region 38 of the chamber 10 and the biologic fluid sample to reside sample region 34 of the chamber 10, without any fluid transfer across the fluid separator in either direction. As will be explained below, in preferred embodiments the ORCS strip portions 20A, 20B on opposite sides of the fluid separator 22 are sufficiently similar; e.g., sufficiently similar so that the same analyte fluid (e.g., a calibration fluid(s) containing the target analyte(s)) when disposed on either side of the fluid separator 22 yields the same optical response during analysis of a given ORCS strip 20. It should be noted, however, that it is not required that the length of a ORCS strip portion 20A, 20B on one side of the fluid separator 22 equal the length of the ORCS strip portion 20B, 20A on the opposite side of the fluid separator 22 since the target analyte analysis may be performed on less than the entire length of an ORCS strip portion. An alternative embodiment of a fluid separator 22 includes one or both of the substrate 12 and the cover sheet 14 including a physical feature (e.g., a trough, or a rib, or the like) that prevents fluid flow in a direction across the separator 22. A trough disposed in one or both of the substrate 12 and the cover sheet 14 may be configured to arrest capillary flow. Alternately, a less preferred method of separating the chamber sample and calibration regions 34, 38 is to physically separate (e.g., cut) them and mount the chamber regions 34, 38 sufficiently far apart such that the respective fluids remain separated during the analysis period.

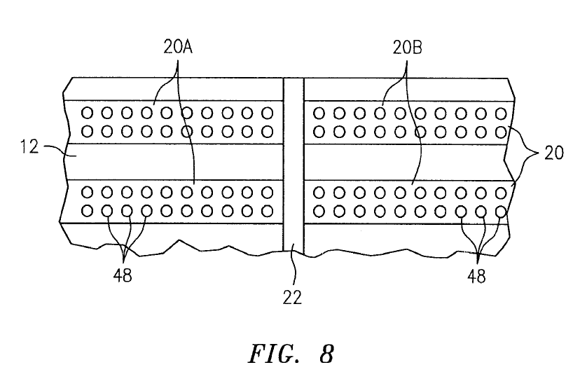

Referring to FIG. 8, in other embodiments an ORCS 20 may be arranged on a chamber substrate surface in a configuration other than the linear configuration described above. For example, discretely formed ORCSs 48 (e.g., bead-like deposits) may be deposited and arranged on a chamber substrate surface in a manner wherein the arrangement of discretely formed ORCSs 48 on one side of a fluid separator 22 is substantially similar to the arrangement of discretely formed ORCSs 48 on the other side of the fluid separator 22. The discretely formed ORCSs 48 may be collectively described as a single ORCS 20. Any arrangement of discrete ORCSs 48 that can be accomplished for both ORCS portions would be acceptable, provided the two ORCS portions 20A, 20B are sufficiently similar so that the same analyte fluid (e.g., a calibration fluid) disposed on either side of the fluid separator 22 would yield the same optical response during analysis for the given ORCS 20. It is not necessary that the discrete ORCSs 48 within a collective ORCS 20 individually have any particular configuration, but it is preferable that they all have at least a substantially similar geometric configuration. In most applications, the characteristics of the ORCSs 20 (e.g., the optode size, constituents, configuration, etc.) are chosen to produce a favorable signal to noise ratio and repeatability of the signal.

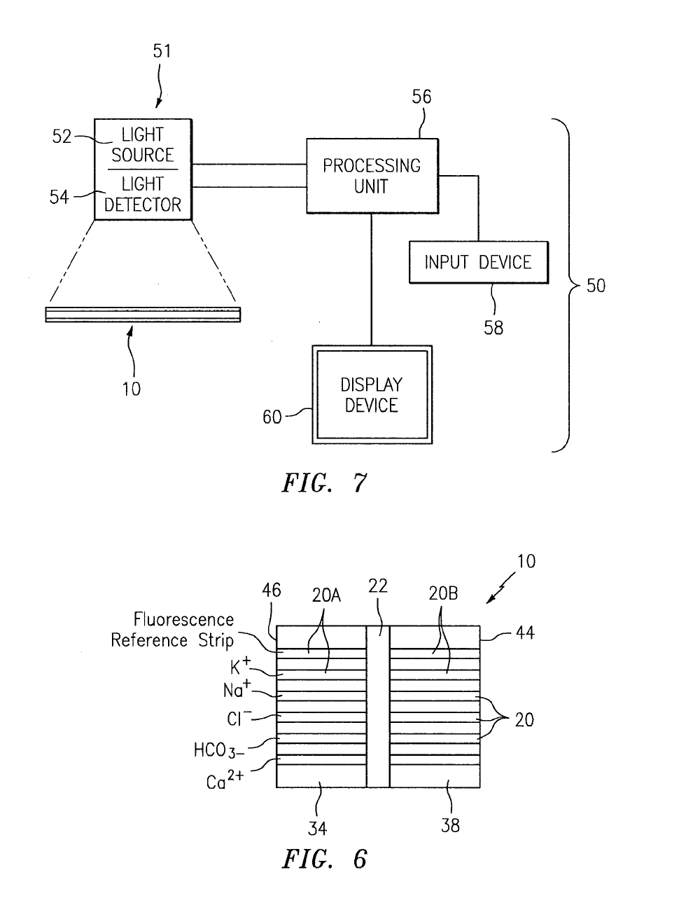

In the case of assays which require only a single color of emission, one or more of the ORCSs 20 may include of a fixed fluorescence reference (e.g., see FIG. 6). The fixed fluorescent reference permits target analyte concentration to be calculated as a function of the ratio of the intensities of two different wavelengths which may be advantageous under certain circumstances. For example, consider that the intensity of the optical response of an ORCS 20 is generally proportional to the volume (e.g., the thickness) of the ORCS 20 being interrogated by light. Consider further that: a) for an ORCS material having a given concentration of particulate optodes, the number of optodes increases with an increase in the ORCS volume; and b) it can be difficult to precisely control the geometry (i.e., the volume) of an ORCS 20 during manufacture of the sensor strip. Hence, variations in the thickness (i.e., the volume) of an ORCS 20 can introduce error in the amount of optical response between portions 20A, 20B of an ORCS 20 being interrogated. To account for this potential variability, a second, fixed fluorescent reference that produces an optical response different from the optical response associated with the target analyte (e.g., the reference emits fluorescent light at first wavelength, and the optodes emit light at a second wavelength) may be used to account for any variability that may be present due to ORCS thickness variability. The target analyte concentration may then be calculated as a function of the ratio of the intensities of the two wavelengths. In most applications, it is believed that the present manner of dividing an ORCS 20 via a fluid separator 22 avoids the need to use a fixed fluorescent reference. In those instances where it is desirable to use one, however, the fixed fluorescent reference may be disposed separately from other sensors, or may be disposed within the ORCS 20.

The present disclosure is not limited to any particular method of disposing an ORCS 20 onto the surface of a substrate. For example, ORCS material may be extruded as a thin filament that is deposited onto the substrate surface, or it may be printed or spread onto the substrate surface, etc. The present disclosure is also not limited to any particular ORCS 20 geometry. For example, in some embodiments, the width of an ORCS 20 may be equal to or less than one millimeter, with adjacent ORCS 20 separated by approximately 0.5 mm. The thickness of an ORCS 20 may, for example, be from 0.1 to 50 microns. An ORCS thickness in the range of about 5 to 20 microns is believed to be particularly useful so that any target analyte/ORCS interaction can come to rapid equilibrium. The length of an ORCS (when in a linear configuration) may be such that the respective portions of the ORCS on either side of the fluid separator may be in the range of about one to ten millimeters (1.0-10.0 mm). ORCS geometries may be optimized to facilitate automated analysis by the analysis device.

To facilitate the sensing of ORCSs within an analysis cartridge, it is preferable that the each ORCS 20 be disposed on a chamber substrate surface 12A, 14A in a known or determinable location. For example, in a chamber 10 configured with a plurality of ORCSs 20 each configured to sense a different analyte (e.g., see FIG. 6), providing information to an analytical device regarding the location and type of ORCS 20 will facilitate the control of the analytical device and the various different analyses. ORCS 20 information (e.g., location, type, etc.) may be communicated to an analytical device, for example, via a bar code or other type label interpretable by a reader portion of the analytical device.

The above described ORCS configurations provide an analysis device 50 (see FIG. 7) with significant manufacturing and quality control advantages. For example, in a mass production environment it is possible that the characteristics of a given type of ORCS 20 may vary from chamber 10 to chamber 10 (e.g., particularly if the chambers are made at different points in time, or different manufacturing runs, etc.). Hence, the analytical results of a highly accurate calibration fluid could vary from chamber 10 to chamber 10 based on manufacturing tolerance. The present disclosure minimizes the potential for such variance by utilizing a single ORCS 20 (e.g., for each type of target analyte, or for each concentration of a target analyte), and separating that particular ORCS 20 into a first portion 20A disposed in the sample region 34 of the chamber 10 and into a second portion 20B disposed in the calibration region 38 of the chamber 10. In other words, the two portions 20A, 20B of a given ORCS 20 are sufficiently similar so that the same analyte fluid (e.g., a calibration fluid) disposed on either side of the fluid separator 22 would yield the same optical response during analysis for the given ORCS 20.

As indicated above, aspects of the present disclosure include one or more systems, apparatus, and methods for performing one or more qualitative and/or quantitative analyte analyses. The present disclosure includes an analysis device that can be used with the above described analysis chamber 10 (and cartridges 26 including an embodiment of the aforesaid chamber 10). An example of such an analysis device 50 is shown diagrammatically in FIG. 7. The analysis device 50 includes a photometric device 51 and at least one processing unit 56. The photometric device includes one or more light sources 52 and one or more light detectors 54. In some embodiments the analysis device 50 may also include an input device 58 (e.g., a key pad, touch screen, etc.) and a display device 60 (e.g., a LCD display, LED display, etc.).

The one or more light sources 52 selectively produce light at one or more wavelengths to which the ORCSs 20 are photometrically sensitive. A non-limited example of a light source 52 is a light emitting diode (LED). In regards to an ORCS being photometrically sensitive (i.e., configured to produce an optical response under certain conditions), an ORCS subjected to the aforesaid wavelengths directly or indirectly produces a first determinable optical response characteristic (e.g., fluorescence or absorbance) in the absence of a target analyte (or with respect to a first chemical environment). The same ORCS 20 in the presence of a sufficient concentration of target analyte (or with respect to a second, different chemical environment) for a period of time sufficient to permit reaction and subjected to the same wavelengths of light produces a second optical response characteristic, which second optical response characteristic is discernibly different from the first optical response characteristic. The change in optical response characteristic of the ORCS 20 are, therefore, indicative of the presence or absence of the target analyte (or chemical environmental change). The specific wavelengths produced and sensed by the analysis device 50 are, therefore, chosen to complement the ORCS 20 used in the analyses. For example, the one or more light sources 52 may include different color LEDs that may be activated for different ORCSs/target analytes. It is envisioned that different wavelength light sources 52 may facilitate color discrimination within the analyses. As an alternative, the one or more light sources 52 may be a source of white light that is utilized with a light filtering system operable to pass predetermined wavelengths of light.

The one or more light detectors 54 are configured to sense light (e.g., at predetermined wavelengths) emitted from, reflected from, or transmitted through the ORCSs 20 wetted by the respective fluids. The present disclosure is not limited to using any particular type or configuration of light detector 54, provided the light detector(s) is adequate for the analysis at hand. An example of an acceptable light detector 54 is a charge couple device (CCD) type image sensor that converts an image of light impinging the sensor into an electronic data format. Complementary metal oxide semiconductor (“CMOS”) type image sensors, fluorometers, and photomultiplier tubes, are examples of other types of light detectors 54 that can be used, and the present disclosure is not limited to any of these examples.

In some embodiments, the one or more light sources 52 and the one or more light detectors 54 may be configured such that no relative movement is required between the light sources/light detectors 52, 54 and the analysis chamber 10; e.g., the analysis device 50 is capable of creating a single image of the calibration region 38, a single image of the sample region 34, or both. In other embodiments, the analysis device 50 is configured such that one of the imaging hardware (e.g., the one or more light sources 52 and the one or more light detectors 54) and the analysis chamber 10 are moved relative to the other. For example, the analysis device 50 may be configured to hold the analysis chamber 10 (e.g., within the analysis cartridge 26) motionless, and the light sources/light detectors 52, 54 may be mounted on a reader head 62 that moves relative to the analysis chamber 10, thereby enabling the light sources/light detectors 52, 54 to “scan” the analysis chamber 10. FIGS. 4 and 5 diagrammatically illustrate an analysis device reader head 62 that moves relative to the cartridge 26. The analysis device 50 is not limited to any particular light source/light detector configuration with respect to an analysis chamber 10 loaded within the analysis device 50; e.g., the light sources/light detectors 52, 54 may be located the same side of an analysis chamber 10 loaded within the analysis device 50, or the light sources/light detectors 52, 54 may be located the opposite sides of an analysis chamber 10 loaded within the analysis device 50, or they may be located remotely with optics (e.g., light pipes) transferring light signals to and from the analysis chamber 10.

In some embodiments, the light detectors and/or the light sources 52, 54 may be packaged in the form of a camera; e.g., the photometric device 51 may be packaged in the form of a camera. For example, if the analysis chamber 10 is configured to be held within a device that fixes the position of the chamber 10, a small fixed-focus camera (e.g., one that uses fluorescent illumination) can be used; e.g., with its lens focused at the surface of an ORCS 20. A Bayer matrix color camera of moderate resolution (5-10 Mpx) is an example of an acceptable camera that can be used to image one or more ORCS 20 (including the calibration region 38 and sample region 34 of each). This type of camera, when used with the usual blocking filter, has sufficient color discrimination to separate the green (560 nm) and red (620-700 nm) fluorescence signals emitted from the common fluorophores. An ORCS 20 may, for example, be interrogated by (generally) blue light (395 nm-470 nm) from one or more LED sources. As indicated above, to further aid color discrimination, different color LEDs may be switched on for different target analytes.

In some embodiments, the present disclosure may utilize a “smart phone” or other communication device that includes a light source and a light detector (e.g., a camera) in combination with a holding device. In such embodiments, the holding device is configured to position (and possibly hold) both the smart phone and a cartridge 26 that includes the analysis chamber in a manner that permits the camera portion of the smartphone to capture an image of the analysis area of the chamber 10. The holding device may include a fixed lens system and a light filter arrangement. For example, a light pipe may be position to align with the smart phone camera flash. The light pipe may be in communication with one or more filters that selectively allow passage of only a certain wavelength(s) of light (e.g., an excitation wavelength), and directs the filtered light to impinge on the analysis area of the chamber. This aspect of the present disclosure is not limited to any particular light filtering arrangement. Also in these embodiments, the smart phone may be configured to include an “app” (e.g., a self-contained software program that may be downloaded to the smart phone) that controls the smart phone in a manner to accomplish the sample imaging, and then permit the sensed light signals to be either analyzed directly (and communicated to the user), and/or sent to a remote site for analysis.

The processing unit 56 may include any type of computing device, computational circuit, or any type of processor or processing circuit capable of executing a series of instructions that are stored (or logic that is stored) in a memory device in communication therewith. The processing unit 56 may include multiple processors and/or multicore CPUs and may include any type of processor, such as a microprocessor, digital signal processor, microcontroller, or the like. The processing unit 56 is configured such that the instructions or logic stored within the memory device is automatically accessed (or selectively accessed via input; e.g., from a user input device) and causes the processing unit 56 to execute the stored instructions in a manner that cause the selected analysis(s) to be performed. The methodologies described herein may faun the basis for the instructions (logic) stored within the memory. The stored instructions may also enable the processing unit 56 to control other aspects of the analysis device 50 such as the one or more light sources 52 and the one or more light detectors 54.

### Operation:

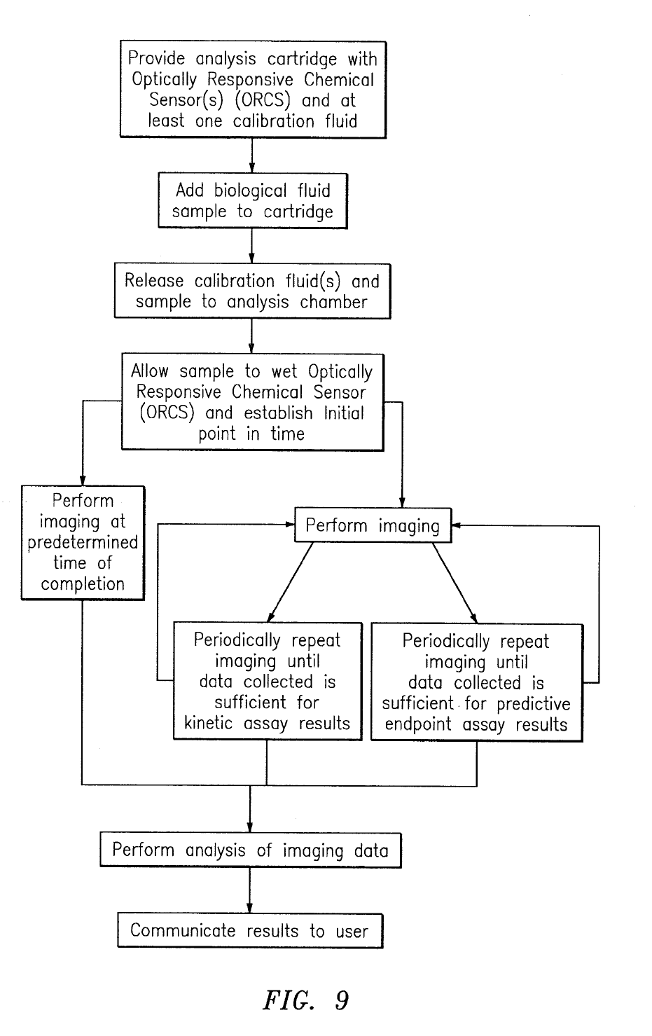

Referring to FIGS. 1-9, to illustrate the utility of the present disclosure, the following example is provided including the sensing of a plurality of target analytes within a biologic fluid sample. The example includes the use of a cartridge 26 having an analysis chamber 10 as described above and shown within FIGS. 1-5. The analysis chamber 10 includes a planar substrate 12 having an inner surface 12A and a cover sheet 14 having an inner surface 14A. The inner surfaces 12A, 14A are separated from one another by a through-plane thickness distance 16 (e.g., about 50 microns is acceptable). FIGS. 4 and 5 show optional spacers 18 disposed between the planar substrate 12 and the cover sheet 14. As stated above, the cartridge 26 includes an analysis chamber 10 having a plurality of ORCSs 20 arranged as lengthwise extending strips disposed on a chamber substrate inner surface. The ORCSs 20 extend lengthwise and each has a substantially constant cross-sectional geometry (e.g., in the Y-Z plane) for substantially the entire length of the ORCS strip 20. Although not absolutely required, for quality control processes, the first ORCS strip may be a fixed fluorescence reference strip (see FIG. 6). The second ORCS strip is configured to sense for the presence of potassium (K⁺) within the biologic fluid sample. The third ORCS strip is configured to sense for the presence of sodium (Na⁺) within the biologic fluid sample. The fourth ORCS strip is configured to sense for the presence of chloride (Cl⁻) within the biologic fluid sample. The fifth ORCS strip is configured to sense for the presence of bicarbonate (HCO₃₋) within the biologic fluid sample. The sixth ORCS strip is configured to sense for the presence of calcium (Ca²⁺) within the biologic fluid sample. A fluid separator 22 in the faun of a hydrophobic barrier material (e.g., Fluoropel™ (Cytonix LLC, MD, USA)) extends widthwise across the ORCS strips 20 at approximately the lengthwise midpoint of the strips. The sample region 34 of the analysis chamber 10 is disposed on one side of the fluid separator 22, and the calibration region 38 is disposed on the opposite side of the fluid separator 22. The ORCS strip portions 20A, 20B on opposite sides of the fluid separator 22 are sufficiently similar (e.g., in composition and geometry) so that an analyte fluid (e.g., a calibration fluid(s) containing the respective analytes) disposed on either side of the fluid separator 22 would yield the same sensed signal during analysis of any of the respective ORCS strips 20.

Referring to FIGS. 4-6, the cartridge 26 includes a biologic fluid sample reservoir 30 fluidly connected to a first fluid passage 32 that includes a fluid control element 40. The first fluid passage 32 is in fluid communication with the sample region 34 of the analysis chamber 10. The fluid control element 40 disposed within the first fluid passage 32 is a capillary stop that prevents fluid passage from the biologic fluid sample reservoir 30 through the first passage 32 and into the sample region 34. The cartridge 26 further includes a calibration fluid reservoir 28 fluidly connected to a second fluid passage 36 that includes a fluid control element 42. The second fluid passage 36 is in fluid communication with the calibration region 38 of the analysis chamber 10. The fluid control element 42 disposed within the second fluid passage 36 is a rupturable membrane that prevents fluid passage from the calibration fluid reservoir 28 through the second fluid passage 36 and into the calibration region 38. The calibration reservoir 28 is filled with a calibration fluid that contains a known concentration of each of the analytes (or a comparable analyte) associated with the ORCS strips 20.

To perform an analysis of the sample, the user places an amount of biologic fluid sample (e.g., anti-coagulated blood, plasma, serum, etc.) within the sample reservoir 30, preferably just prior to performance of the sample analysis. As stated above, aspects of the present disclosure include cartridge 26 configurations wherein multiple point analyses can be performed.

In some instances, it may be useful to image (i.e., interrogate with light and sense for optical response) the portion of the sample region associated with a particular ORCS 20 and the portion of the calibration region associated with the same ORCS 20 prior to the introduction of calibration fluid and/or sample into the analysis chamber 10 to determine initial optical response values. This “pre-analysis” imaging step is not, however, required; e.g., stored optical response values may be utilized.

To initiate the analysis of the sample, the calibration fluid(s) and the sample fluid are passed into the analysis chamber 10; e.g., by opening the flow control devices 40, 42 (e.g., by rupturing a membrane or pushing the sample fluid past a capillary stop) for both sample and calibration fluid reservoirs 30, 28. The respective fluids are drawn out of the respective reservoirs 30, 28 (e.g., by capillary action or by applied pressure) and into the respective sample and calibration regions 34, 38 of the analysis chamber 10. The analysis chamber 10 is configured to allow air disposed within the analysis chamber 10 to escape, and the fluid separator 22 keeps the sample and calibration fluids separate from one another. Note that for samples which are relatively opaque, such as whole blood, it may be preferable to image (i.e., interrogate with light and sense light) the ORCS strips 20 from the outer surface 12B, 14B of the planar substrate 12, 14 on which the ORCS strips 20 are disposed.

Referring to FIG. 7, subsequent to the fluids being drawn into the analysis chamber 10, the analysis device 50 may initiate a timing operation; e.g., an “initial point” in time may be established to correspond with the analysis chamber regions 34, 38 being filled with the respective fluids, or after the analysis device 50 senses that each chamber region 34, 38 is filled with the respective fluid. This example of an “initial point” is an arbitrary example, and the present disclosure is not limited to this example.

For a first type of analysis, the analysis device 50 may be configured to perform the imaging portion of the analyses at a point in time when reactions between the target analytes within the biologic fluid sample and the ORCS 20 (e.g., the optodes disposed within the ORCS), and between the analytes within the calibration fluid and the sensors 20, are sufficiently complete to enable collection of clinically significant data. The term “imaging” as used herein refers to the application of light from the light source(s) 52 to the analysis chamber 10 and the sensing of light emitted from or transmitted through the analysis chamber 10. In particular, the application of light from the light source(s) 52 includes the application of light to an analysis area of the sample region 34 of the analysis chamber 10 and the application of light to the calibration region 38 of the analysis chamber 10. In preferred embodiments of the present disclosure, the light is applied to the analysis areas of both the sample region 34 and the calibration region 38 at or nearly at the same time. Similarly, the sensing of light emitted from or transmitted through (or reflected out of) the analysis chamber 10 includes sensing the analysis area of the sample region 34 of the analysis chamber 10 and sensing the calibration region 38 of the analysis chamber 10. In preferred embodiments of the present disclosure, the sensing of the light emitted from or transmitted through (or reflected out of) the analysis areas of both the sample region 34 and the calibration region 38 is performed at or nearly at the same time. The end point of this period of time may be referred to as the “completion point” and the analyses performed by the processing unit 56 utilize the sensed light data collected at this single “completion point” to perform the analyses.

During the imaging process, the processing unit 56 of the analysis device 50 controls the light sources 52 to illuminate the analysis chamber 10 with the chosen wavelengths and controls the detectors 54 to sense light emitted from or transmitted through (or reflected out of) the analysis chamber 10. As indicated above, the light sources 52 may produce light at a plurality of wavelengths (e.g., light at a peak wavelength that acts as an excitation light source for the optodes within the respective ORCS 20). The light detectors 54 produce signals representative of the light sensed by the light detectors 54, which signals are communicated to the processing unit 56. During the imaging process, the processing unit 56 may also control the positioning of one or both of the analysis chamber 10 and the light sources/light detectors 52, 54. For example, as described above FIGS. 4 and 5 diagrammatically illustrates a portion of an analysis device 50 (i.e., a reader head 62) that is moved traversed relative to a stationary analysis chamber 10 during the imaging process. The interrogation of the ORCS portions 20A, 20B and the light detecting (i.e., light sensing) of the ORCS portions 20A, 20B may be accomplished in a single act (e.g., a single “image”) or it may be done in a manner wherein the ORCS portions 20A, 20B are interrogated and detected independent of one another. As indicated above, the interrogation and detecting of the ORCS portions 20A, 20B, are preferably done at the same time, but it is not required that they be done at the same time.

Once the reaction has reached the completion point, the signals produced by the light detectors 54 are communicated to the processing unit 56. The processing unit 56, which includes instructions (or logic) stored within a memory device, processes the light detector signals using the stored instructions and produces information regarding the target analytes (or change in the chemical environment) within the sample; e.g., the presence or absence of the analytes and/or quantitative information regarding the target analytes, etc.

The instructions include a direct or indirect comparison of the light signals detected from a portion of the sample region 34, 38 associated with a particular ORCS 20 (i.e., the light signals associated with a first portion 20A of the ORCS 20 disposed in the sample region 34) and the light signals detected from a portion of the calibration region 38 associated with the same ORCS 20 (i.e., the light signals associated with a second portion 20B of the ORCS 20 disposed in the calibration region 38). As stated above, the calibration fluid contains a known or ascertainable concentration of the target analyte and will, therefore produce an optical response signal indicative of that concentration of target analyte. The optical response signal associated with the calibration fluid and the second portion 20B of the ORCS 20 can be used, therefore, to confirm the reaction of ORCS 20 (and therefore the optodes disposed within the sensor 20) to the target analyte and/or provide quantitative information with respect to the target analyte. In similar fashion, the optical response signal associated with the sample fluid and the first portion 20A of the ORCS 20 can be used determine the presence or absence of target analyte within the sample and/or provide quantitative information with respect to target analyte within the sample. The comparison of the optical response signals detected from the second portion 20B of the ORCS 20 disposed in the calibration region 38 to the optical response signals detected from the first portion 20A of the ORCS 20 disposed in the sample region 34 can be used to calibrate the analysis and/or to provide quantifiable information. For example, if the relationship between calibration fluid analyte concentration and the magnitude of the ORCS optical response signal is known (e.g., a relationship that can be mathematically described), then the aforesaid signals can be used to quantify the optical response signal values determined in the sample region. This approach may be referred to as a single point approach. As another example, in a case where two or more calibration fluids are used, generally at least one with a concentration higher than that of an expected target analyte concentration range and at least one lower than the expected target analyte concentration range, the ORCS optical response to the concentration of the analyte in calibrator fluids can be used to construct a response curve, whereby the response to analyte concentration can be calculated for any point between the two or more calibrator values. This may be referred to as a multi-point approach. The analysis device 50 may then directly communicate that information to the user (e.g., via a display screen) and/or may transfer that information to a remote location for viewing.

For a second type of analysis, the analysis device 50 may be configured to perform the imaging portion of the analyses at multiple points in time (e.g., periodically) after the sample fluid and the calibration fluid are introduced into the respective regions 34, 38 of the analysis chamber 10; e.g., in the manner described above. The specific number of images that are taken may be selected based on the analyses at hand, and also may depend on the optical response data collected. The type of analyte(s) being evaluated, the type of biologic fluid sample being evaluated, and the type of analysis being performed (e.g., a kinetic analysis or a predictive end-point calculation analysis) all may factor into the number of images taken, the rate at which the images are taken, etc. The analysis device 50 (e.g., the processing unit 56) may be configured to evaluate the imaging variables and select the appropriate imaging variables for the analyses at hand. As indicated above, in preferred embodiments the interrogating light is applied to the analysis areas of both the sample region 34 and the calibration region 38 at or nearly at the same time, and the sensing of the light emitted from or transmitted through (or reflected out of) the analysis areas of both the sample region 34 and the calibration region 38 is performed at or nearly at the same time. Although it is preferable that all readings are taken at the same time, it is not specifically required that the data collection from each region be actually simultaneous as long as the actual time from the initiation of the analysis to the measuring time is recorded and factored into the calculations.

The optical response signals produced by the light detectors 54 may be periodically communicated to the processing unit 56, or may be buffered and collectively communicated to the processing unit 56, or some combination thereof. Similarly, each periodic optical response signal data set may be periodically analyzed by the processing unit 56, or the periodic optical response signal data sets may be buffered and collectively analyzed by the processing unit 56, or some combination thereof. The processing unit 56, which includes instructions (or logic) stored within a memory device, processes the light detector signals using the stored instructions and produces information regarding the target analytes (or chemical environment characteristic) within the sample; e.g., the presence or absence of the analytes and/or quantitative information regarding the target analytes, etc. The analysis device 50 may then directly communicate that information to the user (e.g., via a display device 60) and/or may transfer that information to a remote location for viewing.

The configuration of the ORCS 20 and fluid separator 22 in the analysis chamber 10 of the present disclosure greatly facilitates the possible analyses and eliminates significant potential variability in the results. In the preferred embodiments, one or more ORCSs 20 are disposed on the surface of a substrate and each ORCS 20 is functionally separated into two portions; i.e., a first portion 20A disposed within a sample region 34 of the analysis chamber 10 and another portion 20B disposed within a calibration region 38 of the analysis chamber 10. The two sensor portions 20A, 20B (which were preferably created at the same time, have the same composition, are the same age, and were deposited on the substrate surface at the same time) vary only in their position relative to the fluid separator 22. As a result, variability associated with the ORCS 20 itself between the sample region 34 and the calibration region 38 is insufficient to negatively affect the results of the analysis at hand. Furthermore, because the analysis of the biologic fluid sample can be compared to the known characteristics of the calibration fluid, the specific parameters of the ORCS 20 (e.g., including the optode characteristics) need not be held to tight tolerances. The configuration of the ORCS 20 and fluid separator(s) 22 within the analysis chamber 10 also permits the sample region 34 and the calibration region 38 to be analyzed either simultaneously or very near in time. Hence, any variability in the results that may be affected by a difference in time or temperature between the sample region 34 analysis and the calibration region 38 analysis can be effectively eliminated. Certain of the electronic chip analyses described above utilize a calibration procedure wherein the ion specific electrodes (ISEs) are exposed to a calibration fluid prior to the sample analysis. Once the calibration is performed, the calibration fluid must be removed and typically the analysis chamber housing the ISEs must be washed to remove any residual calibration fluid. This process requires a volume to accept the “used” calibration fluid as well as the wash fluid. This process which operates sequentially also increases the amount of time necessary to perform the analyses. Also, this sequential sensing does not allow the device to use types of chemical reactions which irreversibly alter some of the sensor's reagents.

In some instances it may be desirable to calibrate the analysis device; e.g., to determine the effects of pH on optical response signal intensity. As indicated above, the present disclosure may include an ORCS 20 configured to provide information relating to the pH level of the sample and/or the calibration fluid. In some instances, it may be useful to calibrate the analysis device, for example, to determine the performance characteristics of the analysis device 50. In such instances, the analysis device 50 may be calibrated using a single solution containing all the analytes of interest.

As will be recognized by those of ordinary skill in the pertinent art, numerous modifications and substitutions may be made to the above-described embodiment of the present invention without departing from the scope of the invention as set forth in the appended claims. Accordingly, the preceding portion of this specification is to be taken in an illustrative, as opposed to a limiting sense.

What is claimed is:

1. A method of analyzing a biologic fluid sample for at least one target analyte, comprising:
providing an analysis chamber having at least one optically responsive chemical sensor (ORCS) disposed on a substrate surface, a sample fluid region, a calibration fluid region, and at least one fluid separator fluidically separating the sample fluid region and the calibration fluid region, wherein a first portion of the at least one ORCS is disposed in the sample fluid region and a second portion of the at least one ORCS is disposed in the calibration fluid region;
disposing at least one calibration fluid that includes the target analyte in a known or ascertainable concentration in the calibration fluid region;
disposing the biologic fluid sample in the sample fluid region;
using at least one light source to interrogate the first portion of the ORCS and the second portion of the ORCS with one or more predetermined wavelengths of light, and using the at least one light detector to detect a first optical response from the first portion of the ORCS when interrogated and a second optical response from the second portion of the ORCS when interrogated; and
using a processing unit having at least one processor to analyze the biologic fluid sample relative to the at least one target analyte using the detected first and second optical responses.

2. The method of claim 1, wherein the at least one ORCS includes a plurality of optodes configured to optically respond in the presence of the target analyte and when interrogated with the one or more predetermined wavelengths of light, which plurality of optodes are substantially uniformly distributed in both the first and second portions of the ORCS.

3. The method of claim 2, wherein each of the optodes includes ionophores that selectively interact with the target analyte, chomoionophores that optically respond as a function of protonation, and a matrix.

4. The method of claim 1, wherein the first optical response is a change in at least one of fluorescent emission, absorbance, or reflectance.

5. The method of claim 4, wherein the second optical response is a change in at least one of fluorescent emission, absorbance, or reflectance.

6. The method of claim 5, wherein the first and second optical responses include the optodes emitting fluorescent emissions at a first wavelength when interrogated by the one or more predetermined wavelengths of light and in the absence of the target analyte, and the optodes emitting fluorescent emissions at a second wavelength when interrogated by the one or more predetermined wavelengths of light and in the presence of the target analyte, which second wavelength is different from the first wavelength.

7. The method of claim 1, wherein the at least one light detector produces first signals indicative of the first optical response and second signals indicative of the second optical response, and the processing unit analyzes the biologic fluid sample relative to the at least one target analyte using the first signals and the second signals.

8. The method of claim 1, wherein the analysis chamber includes:
a first ORCS that includes a plurality of first optodes selectively sensitive to a first target analyte, which plurality of first optodes are substantially uniformly distributed in both the first and second portions of the first ORCS; and
a second ORCS that includes a plurality of second optodes selectively sensitive to a second target analyte, which plurality of second optodes are substantially uniformly distributed in both the first and second portions of the second ORCS; and
wherein the first target analyte is different in type from the second target analyte.

9. The method of claim 1, wherein the interrogating and detecting are performed a plurality of times prior to a reaction between the target analyte and the ORCS reaching equilibrium, and the analyzing is a kinetic analysis.

10. The method of claim 1, wherein the interrogating and detecting are performed a plurality of times prior to a reaction between the target analyte and the ORCS reaching equilibrium, and the analyzing is a predictive end-point analysis.

11. An apparatus for analyzing a biologic fluid sample for at least one target analyte, comprising:
an analysis chamber having at least one optically responsive chemical sensor (ORCS) disposed on a substrate surface, a sample fluid region, a calibration fluid region, and at least one fluid separator fluidically separating the sample fluid region and the calibration fluid region, wherein a first portion of the at least one ORCS is disposed in the sample fluid region and a second portion of the ORCS is disposed in the calibration fluid region;
at least one light source and at least one light detector; and
a processing unit having at least one processor, which processing unit is in communication with the at least one light source and the at least one light detector, and in communication with a memory device storing instructions, wherein the instructions when executed cause the processing unit to:
control the at least one light source to interrogate the first portion of the ORCS and the second portion of the ORCS with one or more predetermined wavelengths of light, and control the at least one light detector to detect a first optical response from the first portion of the ORCS when interrogated and a second optical response from the second portion of the ORCS when interrogated; and
analyze the biologic fluid sample using the detected first and second optical responses.

12. The apparatus of claim 11, wherein the at least one ORCS includes a plurality of optodes configured to optically respond in the presence of the target analyte and when interrogated with the one or more wavelengths of light, which plurality of optodes are substantially uniformly distributed in both the first and second portions of the ORCS.

13. The apparatus of claim 12, wherein each of the optodes includes ionophores that selectively interact with the target analyte, chomoionophores that optically respond as a function of protonation, and a matrix.

14. The apparatus of claim 13, wherein the first and second optical responses are a change in at least one of fluorescent emission, absorbance, or reflectance.

15. The apparatus of claim 11, wherein the analysis chamber includes:
a first ORCS that includes a plurality of first optodes selectively sensitive to a first target analyte, which plurality of first optodes are substantially uniformly distributed in both the first and second portions of the first ORCS; and
a second ORCS that includes a plurality of second optodes selectively sensitive to a second target analyte, which plurality of second optodes are substantially uniformly distributed in both the first and second portions of the second ORCS; and
wherein the first target analyte is different in type from the second target analyte.

16. The apparatus of claim 11, wherein the instructions when executed cause the processing unit to control the at least one light source to interrogate, and the at least one light detector to detect, a plurality of times prior to a reaction between the target analyte and the ORCS reaching equilibrium, and to perform a kinetic analysis.

17. The apparatus of claim 11, wherein the instructions when executed cause the processing unit to control the at least one light source to interrogate, and the at least one light detector to detect, a plurality of times prior to a reaction between the target analyte and the ORCS reaching equilibrium, and to perform a predictive end-point analysis.

18. An analysis chamber, comprising:
at least one optically responsive chemical sensor (ORCS) disposed on a substrate surface, which ORCS is configured to optically respond in the presence of a target analyte and when interrogated with one or more predetermined wavelengths of light;
a sample fluid region;
a calibration fluid region; and
at least one fluid separator fluidically separating the sample fluid region and the calibration fluid region;
wherein a first portion of the at least one ORCS is disposed in the sample fluid region and a second portion of the ORCS is disposed in the calibration fluid region.

19. The analysis chamber of claim 18, wherein the at least one ORCS includes a plurality of optodes configured to optically respond in the presence of the target analyte and when interrogated with the one or more wavelengths of light, which plurality of optodes are substantially uniformly distributed in both the first and second portions of the ORCS.

20. The analysis chamber of claim 19, wherein each of the optodes includes ionophores that selectively interact with the target analyte, chomoionophores that optically respond as a function of protonation, and a matrix.

21. The analysis chamber of claim 18, wherein the analysis chamber includes:
a first ORCS that includes a plurality of first optodes selectively sensitive to a first target analyte, which plurality of first optodes are substantially uniformly distributed in both the first and second portions of the first ORCS; and
a second ORCS that includes a plurality of second optodes selectively sensitive to a second target analyte, which plurality of second optodes are substantially uniformly distributed in both the first and second portions of the second ORCS; and
wherein the first target analyte is different in type from the second target analyte.
